(19) United States
(12) Patent Application Publication
(10) Pub. No.: US 2021/0038688 A1
(43) Pub. Date: Feb. 11, 2021

(54) ICAM-1 MARKER AND APPLICATION THEREOF

(71) Applicant: Shanghai Institute of Nutrition and Health, Chinese Academy of Sciences, Shanghai (CN)

(72) Inventors: Yufang Shi, Shanghai (CN); Ying Wang, Shanghai (CN); Chunxing Zheng, Shanghai (CN)

(73) Assignee: Shanghai Institute of Nutrition and Health, Chinese Academy of Sciences, Shanghai (CN)

(21) Appl. No.: 16/965,955

(22) PCT Filed: Jan. 29, 2019
(86) PCT No.: PCT/CN2019/073725

§ 371 Date: Jul. 29, 2020

(30) Foreign Application Priority Data
Jan. 29, 2018 (CN) 201810085799.7

(51) Int. Cl. A61K 38/17; A61K 31/4409; C12N 5/077; G01N 33/68; G01N 33/50; A61K 35/35; A61P 3/04; A61L 27/38; A61L 27/54

(52) U.S. Cl. CPC A61K 38/1774; A61K 31/4409; C12N 5/0653; G01N 33/6893; G01N 33/5005; G01N 2800/044; A61P 3/04; A61L 27/3804; A61L 27/54; C12N 2506/1384; C12N 2501/58; A61K 35/35

(57) ABSTRACT

Provided is an application of an ICAM-1 marker and a regulating agent thereof in promoting or inhibiting differentiation of adipose-derived stem cells into adipose cells, and an application of ICAM-1 or detection reagent thereof in (a) detecting adipose-derived stem cells, and/or (b) determining the risk of a subject suffering from obesity and a corresponding diagnostic kit and method. Further provided is an in vitro non-therapeutic preparation method for fat cells.

### TECHNICAL FIELD

The present invention relates to the field of biotechnology, and more specifically to ICAM-1 and its applications in adipose stem cell recognition and regulation of adipocyte differentiation.

### BACKGROUND

The occurrence of obesity is reflected in the increase of adipose tissue, which includes two effects: hypertrophy of adipocyte , due to excessive intake and accumulation of lipids, and hyperplasia of adipocyte. Mature adipocytes are not capable of mitosis, so that the hyperplasia of adipocytes is caused by the differentiation of fat precursor cells into new adipocytes. Adult adipose tissue is renewed at a rate of 10% per year. In obese population, the elimination rate of adipocytes is no different from that of normal people, but the rate of regeneration is significantly higher than that of normal people, resulting in an hyperplasia of adipocytes. In rodents, it is generally believed that when high-fat diet is used to induce obesity, the size of adipocytes initially increases, and as the time of high-fat feeding goes on, the number of adipocytes gradually increases. Through genetically modified mice in which new adipocytes can be marked, it is found that in the early stages of obesity, adipogenic differentiation is not obvious, while in the later stages, a large number of adipose stem cells differentiate into new adipocytes, especially obviously in visceral adipose tissue. Therefore, obesity is accompanied by the adipogenic differentiation of adipose stem cells, which is an important cause of obesity, whether in humans or rodents. However, the definition of these adipose stem cells and the cellular and molecular regulation mechanisms of their adipogenic differentiation (especially in the obesity stage) are still unclear.

A complete differentiation system in vitro of the differentiation process of adipocytes in vitro and its molecular mechanisms have been established, but it is urgent to study how to regulate the differentiation of adipocytes in vivo. Many scholars have previously confirmed that Sca-1, CD34, CD29, CD24, PDGFR-β and PDGFR-α can label adipose precursor cells, but these markers cannot determine specific adipocyte differentiation types well.

Therefore, there is an urgent need in the art to develop new molecules that can identify adipose stem cells and mark the differentiation of adipocytes.

### SUMMARY OF THE INVENTION

The purpose of the present invention is to provide ICAM-1 and its applications in adipose stem cell recognition and regulation of adipocyte differentiation.

In a first aspect of the present invention, it provides a use of an ICAM-1 inhibitor for preparing a preparation or composition, and the preparation or composition is used for promoting the differentiation of an adipose stem cell into an adipocyte.

In another preferred embodiment, the adipose stem cell is an ICAM-1 positive adipose stromal cell.

In another preferred embodiment, the adipose stem cell is a CD45⁻CD31⁻Sca-1⁻PDGFR-α⁺ICAM-1⁺ cell.

In another preferred embodiment, the adipose stem cell is a CD45⁻CD31⁻ICAM-1⁺ cell.

In another preferred embodiment, the adipose stem cell expresses a regulatory gene for adipogenic differentiation.

In another preferred embodiment, the regulatory gene for adipogenic differentiation is selected from the group consisting of: Pparg, Cebpa, Cebpb, Cebpg, Gata2, Gata3, Irs1, Pparg, Cebba and Fabp4, and a combination thereof.

In another preferred embodiment, the adipose stem cell expresses a characteristic molecule selected from the group consisting of: Sca-1, CD34, CD29, CD24, Pdgfr-β, Zfp423, and a combination thereof.

In another preferred embodiment, the preparation or composition is also used for remodeling of adipose tissues.

In another preferred embodiment, the ICAM-1 inhibitor specifically inhibits the expression or activity of ICAM-1.

In another preferred embodiment, the ICAM-1 inhibitor includes MicroRNA, siRNA, shRNA, and a combination thereof.

In another preferred embodiment, the ICAM-1 inhibitor includes an antibody.

In another preferred embodiment, the ICAM-1 is derived from a human or non-human mammal

In another preferred embodiment, the composition is a pharmaceutical composition.

In another preferred embodiment, the pharmaceutical composition comprises (a) an ICAM-1 inhibitor; and (b) a pharmaceutically acceptable carrier.

In another preferred embodiment, the dosage form of the pharmaceutical composition is an oral dosage form, an injection, or an external pharmaceutical dosage form.

In a second aspect of the present invention, it provides a use of ICAM-1 or a promoter thereof for preparing of a preparation or composition for inhibiting the differentiation of an adipose stem cell into an adipocyte.

In another preferred embodiment, the preparation or composition is used for maintaining the undifferentiated state of the adipose stem cell.

In another preferred embodiment, the ICAM-1 promoter specifically promotes the expression or activity of ICAM-1.

In a third aspect of the present invention, it provides an in vitro non-therapeutic method for preparing an adipocyte, comprising the steps of:

(a) providing an ICAM-1 positive adipose stromal cell;

(b) culturing the adipose stromal cell under conditions suitable for adipocyte differentiation, thereby obtaining a cell population containing a differentiated adipocyte; and

(c) isolating the adipocyte from the cell population.

In another preferred embodiment, the adipose stromal cell is a CD45⁻CD31⁻Sca-1⁻PDGFR-α⁺CAM-1⁺ V cell.

In another preferred embodiment, the adipose stromal cell is a CD45⁻CD31⁻ICAM-1⁺ cell.

In another preferred embodiment, the ICAM-1 positive adipose stromal cell is an adipose stem cell.

In another preferred embodiment, in step (b) and step (c), detecting the expression level of ICAM-1 to determine the degree of differentiation of the adipose stromal cell into the adipocyte in the cell population.

In another preferred embodiment, as the degree of differentiation of the adipose stromal cell into the adipocyte increases, the expression level of ICAM-1 of the adipose stromal cell decreases.

In another preferred embodiment, in step (b), inhibiting the expression of ICAM-1 of the adipose stromal cell, thereby promoting the differentiation of the adipose stromal cell into the adipocyte.

In another preferred embodiment, in step (b), as the culture continues, the expression level of ICAM-1 of the adipose stromal cell gradually decreases.

In another preferred embodiment, in step (b), when the cell population does not substantially express ICAM-1, the adipocyte are isolated from the cell population.

In another preferred embodiment, the substantially no expression means that the ratio N1/N2 of the number of cells N1 expressing ICAM-1 to the total number of cells N2 of the cell population is less than or equal to 5%, preferably less than or equal to 1%.

In a fourth aspect of the present invention, it provides an in vitro non-therapeutic method for inhibiting the differentiation of an adipose stem cell into an adipocyte, comprising maintaining the ICAM-1 expression level of the adipose stem cell.

In another preferred embodiment, the maintenance of the expression level of ICAM-1 includes adding ICAM-1 or a promoter thereof into the adipose stem cell culture system.

In a fifth aspect of the present invention, it provides a use of ICAM-1 or a detection reagent thereof for preparing a detection kit for (a) the detection of an adipose stem cell, and/or (b) the determination of the risk of obesity in a test subject.

In another preferred embodiment, the kit further contains FABP4 or a detection reagent thereof.

In another preferred embodiment, the adipose stem cell has adipogenic differentiation ability.

In another preferred embodiment, the adipose stem cell can differentiate into an adipocyte, resulting in an increase of the number of the adipocyte.

In another preferred embodiment, the detection of an adipose stem cell includes:

(i) testing whether the sample contains an adipose stem cell, and/or

(ii) measuring the number of the adipose stem cell contained in the sample.

In another preferred embodiment, the sample is a tissue sample, preferably, including an adipose tissue, more preferably, an adipose tissue around a blood vessel.

In another preferred embodiment, the kit is used for detecting the proportion of ICAM-1⁺ cells in the sample or detecting the expression level of ICAM-1 of the cells in the sample,thereby detecting the adipose stem cell.

In another preferred embodiment, the determination includes auxiliary determination and/or pre-treatment determination.

In another preferred embodiment, the determination is to compare the ICAM-1⁺ cell proportion A1 of the sample from the test subject with the corresponding ICAM-1⁺ cell proportion A0 of the normal population, and if A1 is significantly higher than A0, it is indicated that the test subject has a high risk of obesity.

In another preferred embodiment, the determination further includes comparing the FABP4⁺ cell proportion B1 of the sample from the test subject with the FABP4⁺ cell proportion B0 of the normal population, and if B1 is significantly lower than B0, it is indicated that the test subject has a high risk of obesity.

In another preferred embodiment, the “significantly higher” means that A1/A0≥1.25, preferably A1/A0≥1.5, more preferably A1/A0≥2.0.

In another preferred embodiment, the “significantly lower” means that B0/B1≥1.25, preferably B0/B1>1.5, more preferably B0/B1≥2.0.

In another preferred embodiment, the number of the normal population is at least 100; preferably at least 300; more preferably at least 500, and most preferably at least 1,000.

In another preferred embodiment, the detection reagent includes a protein chip, a nucleic acid chip, or a combination thereof.

In another preferred embodiment, the detection reagent includes an ICAM-1 specific antibody.

In another preferred embodiment, the ICAM-1 specific antibody is conjugated with or has a detectable label.

In another preferred embodiment, the detectable label is selected from the group consisting of a chromophore, a chemiluminescent group, a fluorophore, an isotope and an enzyme.

In another preferred embodiment, the ICAM-1 specific antibody is a monoclonal antibody or a polyclonal antibody.

In a sixth aspect of the present invention, it provides a diagnostic kit, containing a container, wherein the container contains ICAM-1 or a detection reagent thereof; and a label or instructions, wherein the label or instructions indicate that the kit is used for (a) detecting an adipose stem cell, and/or (b) determining the risk of obesity in the test subject.

In another preferred embodiment, the kit further includes FABP4 or a detection reagent thereof.

In another preferred embodiment, the ICAM-1 and FABP are used as standards.

In another preferred embodiment, the kit further includes a sample pretreatment reagent for testing and instructions.

In another preferred embodiment, the instructions describe the detection method and the determination method based on the A1 value.

In another preferred embodiment, the kit further includes ICAM-1 gene sequences and protein standards.

In a seventh aspect of the present invention, it provides a method for determining the risk of obesity in a test subject, comprising the steps of:

(a) providing a sample from a test subject;

(b) determining the proportion of ICAM-1⁺ cells in the sample as Al;

(c) comparing the A1 in step (b) with the proportion A0, of ICAM-1⁺ cells in the normal population sample, and if A1 is significantly higher than A0, it is indicated that the test subject has a high risk of obesity.

In another preferred embodiment, the method further includes determining the proportion of FABP4⁺ cells B1 in the sample, and comparing B1 with the proportion B0 of FABP4⁺ cells in the normal population, and if B1 is significantly lower than B0, it is indicated that the test subject has a high risk of obesity.

In another preferred embodiment, the test subject is a human or non-human mammal.

In another preferred embodiment, the test sample is a tissue sample, preferably an adipose tissue sample.

In an eighth aspect of the present invention, it provides a use of a stromal cell, which is an ICAM-1 positive stromal cell isolated from an adipose tissue, wherein the stromal cell is used for preparing a cell preparation for the remodeling of an adipose tissue,

and preferably, the remodeling of an adipose tissue includes the remodeling of adipose tissues from the face, buttocks, and breasts.

In another preferred embodiment, the remodeling includes adipose tissue remodeling in cosmetic applications and adipose tissue remodeling in wound healing.

In another preferred embodiment, the remodeling includes adipose tissue filling.

In another preferred embodiment, the cosmetology includes the cosmetology of the face, the waist, the legs, the breasts, the hands, and the neck.

In another preferred embodiment, the remodeling further includes adipose tissue fillings in cosmetic, bodybuilding, and plastic surgery applications.

In another preferred embodiment, the cosmetology includes the filling of an adipose tissue, and the overall cosmetic, bodybuilding, and plastic effects brought by the filling of adipose tissue.

In another preferred embodiment, the preparation further includes: an ICAM-1 inhibitor.

It should be understood that, within the scope of the present invention, the technical features specifically described above and below (such as the Examples) can be combined with each other, thereby constituting a new or preferred technical solution which needs not be described one by one.

### DESCRIPTION OF DRAWINGS

FIG. 1 shows that ICAM-1⁺ adipose stromal cells have the potential to differentiate into adipose stem cells. Specifically, flow cytometry is used to sort CD31⁻CD45⁻adipose stromal cells from the visceral adipose tissue for single cell analysis.

FIG. 1A shows the analysis of the expressions of Sca-1 and PDGFR-α in CD31⁻CD45⁻adipose stromal cell in visceral adipose tissue using flow cytometry.

FIG. 1B shows the analysis of the expression levels of ICAM-1 in CD31⁻CD45⁻Sca-1⁺PDGFR-α⁺ cell population in visceral adipose tissue (epididymal adipose) and subcutaneous adipose tissue (groin fat) using flow cytometry.

FIG. 1C shows the sorting of ICAM-1⁺ and ICAM-1⁻ cells in the CD31⁻CD45⁻Sca-1⁺PDGFR-α⁺ cell population, and the adipogenic differentiation and the expression levels of adipose stem cell-related genes detected by real-time PCR.

FIG. 1D shows the spontaneous adipocyte differentiation observed during the co-cultivation of ICAM-1⁻ cells isolated from the CD31⁻CD45⁻Sca-1⁺PDGFR-α⁺ cell population in the adipose tissue of wild mice and the ICAM-1⁺ cells isolated from CD31⁻CD45⁻Sca-1⁺PDGFR-α⁺ cell population in the GFP mouse adipose tissue.

FIG. 2 shows the in vivo adipogenic differentiation of ICAM-1⁺ adipose stem cells.

FIG. 2A shows the construction of ICAM-1 adipose stem cell tracer mice by the hybridization of mTmG mice with the Icaml-CreERT2 knock-in mice, and the activation of the recombinase by tamoxifen.

FIG. 2B shows the condition of adipocytes produced by ICAM-1⁺ adipose stem cells during the development of adipose tissue in newborn mice using the whole fluorescence staining technique of adipose tissue.

FIG. 2C shows the condition of adipocytes produced by ICAM-1⁺ adipose stem cells during the obesity induced by high-fat diet using the whole fluorescence staining technique of adipose tissue.

FIG. 3 shows the evolution characteristics of ICAM-1⁺ adipose stem cells under obesity conditions.

FIG. 3A shows the construction of Fabp4-Cre; mTmG mice to study adipose stem cells during adipogenic differentiation.

FIG. 3B shows the expression level of ICAM-1 in adipose precursor cells in a state of adipogenic differentiation in adipose tissue using flow cytometry.

FIG. 3C shows the FABP4 (EGFP-labeled) expression levels of ICAM-1⁺ adipose stem cells in visceral adipose tissue and subcutaneous adipose tissue under normal diet and high fat-induced obesity using flow cytometry.

FIGS. 3D and 3E are repeated statistical analyses of the experiment as shown in FIG. 3.

FIG. 3F shows the sorting and obtaining of adipocytes (Adi), ICAM-⁺EGFP⁺(I³⁰ G⁺), ICAM-1EGFP⁻(I⁺G⁻) and ICAM-1⁻(I⁻) cells, and the transcriptome analysis and correlation analysis thereof, using flow cytometry.

FIG. 3G shows transcriptome analysis of the differentially expressed genes of mature adipocytes, I⁺EG⁺cells, I⁺G⁻cells and I⁻cells, mainly relating to PPAR signaling, the formation and absorption of fat, fatty acid biosynthesis and fatty acid elongation.

FIG. 4 shows that ICAM-1 negatively regulates the directed differentiation of adipose stem cells.

FIG. 4A shows the body weight changes of wild-type (WT) mice and ICAM-1^{-/-} mice under normal diet and high-fat diet.

FIG. 4B shows the changes in adipose tissue weight of wild-type (WT) mice and ICAM-1^{-/-} mice under normal diet and high-fat diet.

FIG. 4C shows the results of fluorescence staining analysis for the size of adipocyte in adipose tissues.

FIG. 4D shows the statistical results of the sizes of adipocyte in adipose tissues analyzed by fluorescence staining.

FIGS. 4E-4H show the changes in mice body weight (FIG. 4E) and adipose tissue weight (FIG. 4F) at different time points observed, and the sizes of adipocyte in adipose tissues by fluorescent staining (FIG. 4G) and statistical analysis thereof (FIG. 4H), in the irradiated WT mice and ICAM-1^{-/-} mice after transplantation of bone marrow from WT mice for bone marrow reconstitution and with a high-fat diet given.

FIG. 4I shows analysis of the production of adipocytes by adipose stem cells under the condition of ICAM-1 deletion after the hybridization of ICAM-1^{-/-} mice and ICAM-1^{-/-} mice with Fabp4-Cre; mTmG mice, and the statistical analysis thereof, using flow cytometry.

FIG. 4J shows the statistical analysis of multiple mice by flow cytometry analysis as shown in 4I.

FIG. 4K shows the western blot analysis of the GFP content in adipose tissue stromal cells to clarify the new condition of the adipocyte.

FIG. 5 shows that ICAM-1 negatively regulates the adipogenic differentiation of adipose stem cells.

FIGS. 5A-5D show the expressions of genes related to adipogenic differentiation detected at different times, including Pparg (FIG. 5A) and Ceppa (FIG. 5B), Fabp4 (FIG. 5C), Plin1 (FIG. 5D), after the separation and adipogenic differentiation of adipose stem cells from WT mice and ICAM-1^{-/-} mice.

FIG. 6 shows that ICAM-1 negatively regulates the directed differentiation of adipose stem cells through Rho GTPase.

FIG. 6A shows the expression levels of Rho-GTP, Rho-GDP and total Rho in adipose stem cells derived from WT mice and ICAM-1^{-/-} mice detected using active Rho GTPases pull-down assay.

FIG. 6B shows the results of F-actin cytoskeleton staining.

FIG. 6C shows the adipogenic differentiation of adipose stem cells observed using oil red staining, wherein DMSO or 10 μM Y-27632 (ROCK inhibitor) is added respectively during the in vitro differentiation of adipose stem cells derived from WT mice and ICAM-1^{-/-} mice.

FIG. 6D shows Western blot detection of Perilipin A protein expression after adipogenic differentiation of WT and ICAM-1^{-/-} mice-derived adipose stem cells under Y-27623 or DMSO treatment.

FIG. 6E and FIG. 6F respectively show the mRNA levels of Pparg (FIG. 6E) and Fabp4 (FIG. 6F) after adipogenic differentiation of WT and ICAM-1^{-/-} mice-derived adipose stem cells under Y-27623 or DMSO treatment, using Real time PCR.

FIG. 6G shows the adipogenic differentiation of adipose stem cells observed using oil red staining, wherein RA2 (Rho agonist) is added during the in vitro differentiation of adipose stem cells derived from WT mice and ICAM-1^{-/-} mice.

FIGS. 6H-6K respectively shows the mRNA levels of Pparg (FIG. 6H), Cebpa (FIG. 61), Fabp4 (FIG. 6J) and Plin1 (FIG. 6K) detected using Real time PCR method.

FIG. 6L-6N respectively show the visual observation result (FIG. 6L), adipose tissue observation result (FIG. 6M) and statistical analysis of changes in subcutaneous adipose tissue weight (6N) in the right subcutaneous adipose tissue of WT mice and ICAM-1^{-/-} mice that were given a high-fat diet induced obesity is injected with RA2 (every 2 days, 0.5 ii g).

FIG. 7 shows the role of ICAM-1 in the recognition and regulation of human adipose stem cells.

FIG. 7A shows the ICAM-1 expression in human adipose tissue adipose precursor cells detected using flow cytometry.

FIG. 7B shows the tissue localization of ICAM-1⁺ adipose stem cells in human adipose tissue by immunofluorescence detection.

FIG. 7C shows the expression changes of ICAM-1 and FABP4 in the process of adipogenic differentiation of adipose stem cells detected by Real time PCR.

FIG. 7D shows the expression of ICAM-1 in human adipose stem cells knocked down by ICAM-1 siRNA.

FIG. 7E shows the adipogenic differentiation ability of the cells observed by oil red staining after the expression of ICAM-1 of human adipose stem cells knocked down with ICAM-1 siRNA.

FIGS. 7F-7G respectively show the adipogenic differentiation-related gene expression and Rho GTP activity of adipose stem cells during adipogenic differentiation with the expression of ICAM-1 interfered.

FIGS. 7H-7J respectively show the expressions of adipogenic differentiation-related genes (PPARG, CEBPA, FABP4) in adipose stem cells observed with RA2 activated Rho after interfering with ICAM-1 expression.

FIGS. 7K-7L respectively show the correlation analysis results using human adipose tissue as specimens with the body fat ratio BMI index, the expression intensity of ICAM-1, and the expression level of Fabp4⁺ preadipocytes in CD31⁻CD45⁻ adipose stromal cells.

### DETAILED DESCRIPTION OF INVENTION

After extensive and intensive researches, the inventors have for the first time unexpectedly discovered a new molecule for identifying adipose stem cells. Specifically, the present invention provides an application of ICAM-1 and its regulators in promoting or inhibiting the differentiation of adipose stem cells into adipocytes, and an application of ICAM-1 or its detection reagents in (a) detecting adipose stem cells, and/or (b) determining the risk of obesity of test subjects, and corresponding diagnostic kits and methods. The present invention also provides an in vitro non-therapeutic method for preparing adipocytes. Experiments have shown that ICAM-1⁺ adipose stem cells are located around the blood vessels of adipose tissue and have the ability to spontaneously differentiate into adipocytes. They can differentiate into adipocytes in both in vitro and in vivo experiments and participate in the development and remodeling of adipose tissue. In addition, the number of ICAM-1⁺ adipose stem cells is proportional to the enlargement and increase of obese adipose tissue, which can be used to guide the diagnosis of obesity. The present invention is completed on this basis.

Terms

As used herein, the terms “targeted preadipocytes” and “preadipocytes” refer to mesenchymal stem cells that begin to lose pluripotency in adipose tissue and become precursor cells that can differentiate into adipocytes.

As used herein, the term “stromal reserve cells” refers to a type of adipose stromal cells whose differentiation characteristics are not clear. They may have a certain adipogenic differentiation potential but this potential is lower than that of preadipocytes.

As used herein, the term “adipose stromal cells” refers to a type of cells with many characteristics of mesenchymal stem cells that are non-blood cells and non-endothelial cells in adipose tissue

As used herein, the term “adipose stem cells” refers to stem cells that are able to differentiate into adipocytes.

ICAM-1

ICAM-1 (Intercellular adhesion molecule-1, ICAM-1, CD54) is a cell surface adhesion molecule that has attracted much attention. It is a type I transmembrane protein, and its molecular weight varies from 80 to 114 kDa depending on its degree of glycosylation, and the molecular weight of unglycosylated ICAM-1 is 60 kDa (38). The extracellular part of ICAM-1 contains 453 amino acids, mainly hydrophobic amino acids, forming five immunoglobulin (Immunoglobulin, Ig)-like domains. The extracellular part is connected to a very short (comprising 28 amino acids) cytoplasmic tail through a hydrophobic transmembrane region containing 24 amino acids. Its cytoplasmic tail lacks the classic signaling motif, but it has a tyrosine residue that may play an important role in its signaling. The gene sequence of ICAM-1 contains 7 exons with exon 1 encoding a signal peptide, exons 2-6 respectively encoding one of the five Ig domains, and exon 7 encoding the transmembrane region and the cytoplasmic region tail.

ICAM-1 ligands include β2 integrins LFA-1 (CD11a/CD18) and Mac-1 (CD11b/CD18), fibrinogen, and rhinoviruses on leukocytes.

ICAM-1 plays an important role in both innate immunity and acquired immune response. It mediates leukocytes through the blood vessel wall to enter the inflammation site, and also regulates the interaction between antigen presenting cells (APCs) and T cells, and participates in the immunological synapse formation. ICAM-1 can transmit signals from outside to inside. The cytoplasmic tail of ICAM-1 is only 28 amino acid long, and lacks the known kinase activity and protein interaction domain that can recruit downstream signal molecules. But it has many positively charged amino acids and a tyrosine residue (Y512). At present, many signaling molecules and adaptor proteins associated with the ICAM-1 pathway have been found in different cells, especially the actin-cytoskeleton related molecules, including a-actinin, ERM protein, cortactin, and β-tubulin. In B cells, ICAM-1 cross-linking can activate Src family kinases, such as p53/p56 Lyn. A very important molecule in the ICAM-1 signaling pathway is the small GTPase Rho, a member of the Ras superfamily of G proteins. Rho and the downstream Rho associated kinase (ROCK) play an important role in regulating cytoskeleton rearrangement and maintaining cell morphology. Cross-linking with antibodies or co-cultivation with monocytes can induce clustering of ICAM-1, along with co-localization of ERM proteins and assembly of tension fibers. This process requires the activation of RhoA, and the ICAM-1 cytoplasmic tail plays an important role in this process: clusters of ICAM-1 without the cytoplasmic tail cannot activate Rho protein. The activation and inactivation of Rho are strictly regulated by many factors, including guanine exchange factors (GEFs), GTPase activating proteins (GAPs) and guanine nucleotide dissociation inhibitors (GDI). The specific mechanism by which ICAM-1 activates Rho is unclear, but ERM protein and Rho-GDI may play an important role in it. On endothelial cells, ICAM-1 binds to LFA-1 or Mac-1 on leukocytes to activate downstream Rho and

ROCK, causing cytoskeletal rearrangement and morphological changes, thereby mediating leukocytes going through blood vessels and entering inflammatory tissues.

The ICAMI-1 positive adipose stromal cells obtained by sorting can be used for medical cosmetology, such as adipose tissue remodeling.

ICAM-1 Inhibitors and Promoters

The present invention provides the application of ICAM-1 inhibitors in promoting the differentiation of adipose stem cells into adipocytes, and the application of ICAM-1 or its promoters in inhibiting the differentiation of adipose stem cells into adipocytes. Wherein, the ICAM-1 inhibitor specifically inhibits the expression or activity of ICAM-1, and the ICAM-1 promoter specifically promotes the expression or activity of ICAM-1.

Based on the above application, the present invention also provides an in vitro non-therapeutic method for preparing adipocytes, comprising the steps of:

(a) providing an ICAM-1 positive adipose stromal cell;

(b) culturing the adipose stromal cell under conditions suitable for adipocyte differentiation, thereby obtaining a cell population containing a differentiated adipocyte; and

(c) isolating the adipocyte from the cell population.

RNA Interference (RNAi)

In the present invention, one type of effective ICAM-1 inhibitor is interfering RNA.

As used herein, the term “RNA interference (RNAi)” refers to: the processes in which some small double-stranded RNA can efficiently and specifically block the expression of specific genes in vivo, promote mRNA degradation, and induce cells to show a phenotype with specific gene deletions, which is also called RNA intervention or RNA interference. RNA interference is a highly specific gene silencing mechanism at the mRNA level.

As used herein, the term “small interfering RNA (siRNA)” refers to a short double-stranded RNA molecule that can target mRNA with homologous complementary sequences to degrade specific mRNA. This process is the RNA interference pathway.

In the present invention, interfering RNA includes siRNA, shRNA and corresponding constructs.

A typical construct is double-stranded, and its positive or negative strand contains the structure as shown in Formula I:

Seq_{forward}-X-Seqᵣₑᵥₑᵣₛₑ Formula I

wherein,

Seq_{forward} is a nucleotide sequence of ICAM-1 gene or fragment;

Seqᵣₑᵥₑᵣₛₑ is a nucleotide sequence basically complementary to the Seq forward;

X is an spacer sequence located between the Seq_{forward} and the Seqᵣₑᵥₑᵣₛₑ, and the spacer sequence is not complementary to the Seq_{forward} and the Seqᵣₑᵥₑᵣₛₑ.

In a preferred embodiment of the present invention, the lengths of Seq_{forward} and Seqᵣₑᵥₑᵣₛₑ are 19-30 bp, preferably 20-25 bp.

In the present invention, a typical shRNA is shown in Formula II,

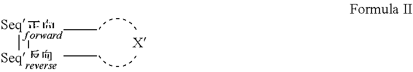

wherein,

Seq′_{forward} is the RNA sequence or sequence fragment corresponding to the Seq forward sequence;

Seq′ᵣₑᵥₑᵣₛₑ is a sequence that is basically complementary to the Seq′_{forward};

X′ is none; or is an spacer sequence located between Seq′_{forward} and Seq′ᵣₑᵥₑᵣₛₑ, and the spacer sequence is not complementary to Seq′_{forward} and Seq′ᵣₑᵥₑᵣₛₑ,

∥ represents the hydrogen bond formed between Seq_{forward} and Seqᵣₑᵥₑᵣₛₑ.

In another preferred embodiment, the length of the spacer sequence X is 3-30 bp, preferably 4-20 bp.

Wherein, the target genes targeted by the Seq forward sequence include (but are not limited to): Beclin-1, LC3B, ATGS, ATG12, or a combination thereof.

Composition and Method of Administration

The present invention also provides a composition for promoting or inhibiting the differentiation of adipose stem cells into adipocytes containing an ICAM-1 inhibitor or promoter as an active ingredient. The composition includes (but is not limited to): a pharmaceutical composition, a food composition, a dietary supplement, a beverage composition and the like.

In the present invention, the ICAM-1 inhibitor can be directly used for medical cosmetology, for example, for the remodeling of adipocytes. When using the ICAM-1 inhibitor of the present invention, other components can also be used at the same time, such as used together with adipose stem cells.

The present invention also provides a pharmaceutical composition, which contains a safe and effective amount of the ICAM-1 inhibitor or promoter of the present invention and a pharmaceutically acceptable carrier or excipient. Such carriers include (but are not limited to): saline, buffer, glucose, water, glycerol, ethanol, powder, and a combination thereof. The pharmaceutical preparation should match the mode of administration. The pharmaceutical composition of the present invention can be prepared in the form of injection, for example, prepared by conventional methods with physiological saline or an aqueous solution containing glucose and other adjuvants. Pharmaceutical compositions such as tablets and capsules can be prepared by conventional methods. Pharmaceutical compositions such as injections, solutions, tablets and capsules should be manufactured under sterile conditions. The pharmaceutical combination of the present invention can also be made into a powder for inhalation. The dosage of the active ingredient is a therapeutically effective amount, for example, about 1 microgram/kg body weight to about 5 mg/kg body weight per day. In addition, the ICAM-1 inhibitor of the present invention can also be used together with other therapeutic agents.

The pharmaceutical composition of the present invention can be administered to subjects in need (such as humans and non-human mammals) in a conventional manner. Representative administration methods include (but are not limited to): oral, injection, aerosol inhalation, and the like.

When using the pharmaceutical composition, a safe and effective amount of an ICAM-1 inhibitor is administered to a mammal, wherein the safe and effective amount is usually at least about 10 micrograms/kg body weight, and in most cases, no more than about 8 mg/kg body weight, and preferably, the dosage is about 10 micrograms/kg body weight to about 1 mg/kg body weight. Of course, the specific dosage should also consider factors such as the route of administration, the patient's health status, etc., which are within the skill range of a skilled physician.

Detection Reagent

The detection reagent of the present invention includes a protein chip, a nucleic acid chip, and a combination thereof.

In another preferred embodiment, the detection reagent of the present invention further includes an ICAM-1 specific antibody.

Protein chip is a high-throughput monitoring system that monitors the interaction between protein molecules through the interaction of target molecules and capture molecules. Capture molecules are generally pre-immobilized on the surface of the chip. Because of the high specificity and strong binding to antigens of antibodies, they are widely used as capture molecules. For research on protein chips, it is very important to effectively immobilize antibodies on the surface of the chip, and especially in terms of the consistency of the immobilized antibodies, it is very important to enhance the sensitivity of the protein chip. G protein is an antibody binding protein, which specifically binds to the FC fragment of the antibody, so it has been widely used to immobilize different types of antibodies. The protein chip for detecting ICAM-1 of the present invention can be prepared by various techniques known to those skilled in the art.

Nucleic acid chips, also known as DNA chips, gene chips or gene microarrays, refer to the in-situ synthesis of oligonucleotides on a solid support or a large number of DNA probes are directly solidified on the surface of the support in an orderly manner by microprinting, and then hybridized with the labeled sample. Through the detection and analysis of the hybridization signal, the genetic information of the sample can be obtained. In other words, the gene chip is obtained using micro-processing technology to regularly arrange and fix tens of thousands or even millions of DNA fragments (gene probes) with specific sequences on a 2 cm² silicon chip, glass slide and other supports. It forms a two-dimensional DNA probe array, which is very similar to the electronic chip on an electronic computer, so it is called a gene chip.

The present invention relates to polyclonal antibodies and monoclonal antibodies specific to human ICAM-1, especially monoclonal antibodies. Here, “specificity” means that the antibody can bind to human ICAM-1 gene product or fragment. Preferably, it refers to those antibodies that can bind to human ICAM-1 gene products or fragments but do not recognize and bind to other unrelated antigen molecules. The antibodies in the present invention include those molecules that can bind to and inhibit the human ICAM-1 protein, as well as those antibodies that do not affect the function of the human ICAM-1 protein. The present invention also includes those antibodies that can bind to the human ICAM-1 gene product in modified or unmodified forms.

The present invention not only includes complete monoclonal or polyclonal antibodies, but also includes immunologically active antibody fragments, such as

Fab′ or (Fab) 2 fragments; heavy chains of antibodies; light chains of antibodies; genetically engineered single-chain Fv molecules (Ladner et al., U.S. Pat. No. 4,946,778); or chimeric antibodies, such as antibodies that have the binding specificity of a murine antibody but still retain the portion of the antibody from human.

The antibody of the present invention can be prepared by various techniques known to those skilled in the art. For example, the purified human ICAM-1 gene product or its antigenic fragments can be administered to animals to induce the production of polyclonal antibodies. Similarly, cells expressing human ICAM-1 protein or its antigenic fragments can be used to immunize animals to produce antibodies. The antibody of the present invention may also be a monoclonal antibody. Such monoclonal antibodies can be prepared using hybridoma technology (see Kohler et al., Nature 256; 495, 1975; Kohler et al., Eur. J. Immunol. 6: 511, 1976; Kohler et al., Eur. J. Immunol. 6:292, 1976; Hammerling et al., In Monoclonal Antibodies and T Cell Hybridomas, Elsevier, NY, 1981). The antibodies of the present invention include antibodies that can block the function of human ICAM-1 protein and antibodies that do not affect the function of human ICAM-1 protein. The various antibodies of the present invention can be obtained by using fragments or functional regions of the human ICAM-1 gene product through conventional immunization techniques. These fragments or functional regions can be prepared by recombinant methods or synthesized by a peptide synthesizer. Antibodies that bind to the unmodified form of human ICAM-1 gene products can be produced by immunizing animals with gene products produced in a prokaryotic cell (such as E. Coli); antibodies that bind to post-translationally modified forms (such as a glycosylated or phosphorylated protein or polypeptide) can be obtained by immunizing animals with gene products produced in a eukaryotic cell (such as a yeast or insect cell).

Detection Method and Detection Kit

The present invention provides a detection method and a detection kit using ICAM-1 and a detection reagent thereof.

Specifically, the present invention provides a kit, containing a container, wherein the container contains ICAM-1 or a detection reagent thereof; and a label or instructions, the label or instructions indicate that the kit is used for (a) detecting an adipose stem cell, and/or (b) determining the risk of obesity in the test subject.

The present invention also provides a method for determining the risk of obesity of a test subject, including the steps of:

(a) providing a sample from a test subject;

(b) determining the proportion of ICAM-1⁺ cells in the sample as A1;

(c) comparing the A1 in step (b) with the proportion A0, of a ICAM-1⁺ cells in the normal population sample, and if A1 is significantly higher than A0, it is indicated that the test subject has a high risk of obesity.

In another preferred embodiment, the method further includes determining the proportion of FABP4⁺ cells B1 in the sample, and comparing B1 with the proportion B0 of FABP4⁺ cells in the normal population, and if B1 is significantly lower than B0, it is indicated that the test subject has a high risk of obesity.

The main advantages of the present invention include:

(a) The present invention has found that ICAM-1⁺ adipose stem cells have the ability to spontaneously differentiate into adipocytes, and can differentiate into adipocytes in both in vitro and in vivo experiments and participate in the development and remodeling of adipose tissues.

(b) The present invention has found that the number of ICAM-1⁺ adipose stem cells is proportional to the enlargement and increase of obese adipose tissues, and can be used for guiding the diagnosis of obesity.

(c) The present invention has found that ICAM-1 has a negative regulatory effect on the in vivo adipogenic differentiation of human preadipocytes, and the expression level of ICAM1 in human preadipocytes gradually decreases with adipogenic differentiation.

The present invention will be further explained below in conjunction with specific embodiments. It should be understood that these embodiments are only used to illustrate the present invention and not to limit the scope of the present invention. The experimental methods that do not indicate specific conditions in the following examples usually follow the conventional conditions or the conditions recommended by the manufacturer. Unless otherwise stated, percentages and parts are calculated by weight.

General Materials and Methods

ICAM-1^{-/-} mice (B6.129S4-Icamltmllcgr/J) were purchased from Jackson Laboratory (Bar Harbor, ME, USA). Fabp4-Cre (B6.Cg-Tg(Fabp4-cre)1Rev/JNju) mice, mTmG (B6.129(Cg)-Gt(ROSA)26Sortm4(ACTB-tdTomato,-EGFP)Luo/JNju) mice were purchased From Nanjing Institute of Model Animals. The Icaml-CreERT2 knockin mice were constructed by the Southern Model Biology Center, wherein the CreERT2 expression sequence was directly inserted into the start codon ATG of the Icaml gene using Cas9 technology.

Tamoxifen Induces Cell Lineage Tracing In Vivo

After birth, Icam-l-CreRET2; mTmG mice were intraperitoneally injected with 200 μg/mice Tamoxifen from P1 to P3 for 3 consecutive days. Tamoxifen was formulated with corn oil as a mother liquor of 20 mg/ml. After 4-6 weeks, the mice were euthanized and adipose tissue was analyzed for detection of EGFP+adipocytes.

The Detection of Body Fat Ratio In Mice

Obesity mice induced by high-fat were used for detecting adipose tissue and other “lean” tissues with the Body Composition Analyzer. The data were measured for each mouse 2-3 times and the average was taken.

Cultivation of adipose stromal cells The sorted CD31⁻CD45⁻Sca-1⁺PDGFR-α⁺ adipose stromal cells as well as CD31⁻CD45⁻Sca-1³⁰ PDGFR-α⁺ICAM-1⁺ and CD31⁻CD45⁻Sca-1³⁰ PDGFR-α³⁰ICAM-1⁻ and other components were cultured in DMEM low-sugar medium supplemented with 10% FBS alone or in combination. In some experiments, adherent adipose mononuclear cells were directly cultured in DMEM low-sugar medium supplemented with 10% FBS, and immune cells and vascular endothelial cells were removed during the medium exchange and passage steps to obtain simple adipose stromal cells.

Induction of Adipogenic Differentiation of Stromal Cells

For preparing differentiation medium, 0.5 mM 3-isobutyl-1-methylxanthine (IBMX), 50 μM indomethacin, 10 μg/ml insulin and 0.5 μM dexamethasone were added to 10% FBS of DMEM high glucose medium. When the adipose stromal cells grew to 100% confluence, the differentiation medium was changed and the medium was changed every two days, and it was taken about 5 days until differentiation was completed.

### EXAMPLE 1

Adipose Stromal Cells Expressing ICAM-1 Were Potential Adipose Stem Cells

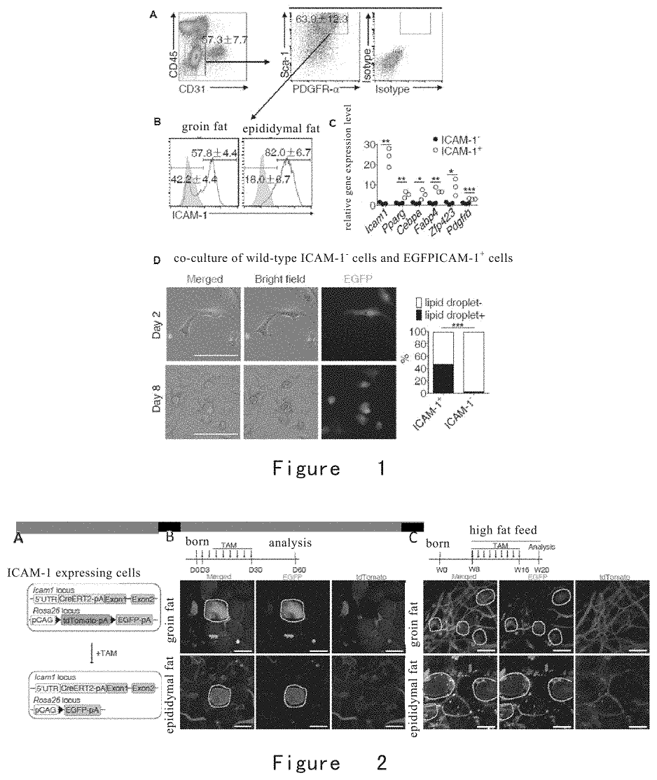

By analyzing the cell characteristics of non-endothelial cells and non-leukocytes (CD31⁻CD45⁻ cells) in adipose tissues rich in adipose stem cells, it is found that most of the CD31⁻CD45⁻stromal cells were CD34⁺ and CD29⁺, which were also PDGFR-α⁺ Sca-1⁺ (FIG. 1A), and the latter were two characteristic surface molecules of mesenchymal stromal cells (MSCs).

Furthermore, the adipose stromal cells were analyzed by flow cytometry, and it was found that most of the CD45⁻CD31⁻ stromal cells were Sca-1⁺DGFR-α⁺. This cell group can be divided into two groups: ICAM-1⁺ and ICAM-1⁻. We found that about 50% of this group ofCD45⁻CD31⁻Sca-1⁻PDGFR-α⁺ cells in the groin adipose tissue (subcutaneous adipose tissue) were ICAM-1 positive, and about 80% of the epididymal adipose tissue (visceral adipose tissue) was ICAM-1 positive (FIG. 1B).

By flow cytometry sorting, two groups of stromal cells, CD45⁻CD31⁻Sca-1⁻PDGFR-α⁺ICAM-1⁺ and CD45⁻CD31⁻Sca-1³⁰ PDGFR-α⁺ICAM-1⁻, were obtained and genetic analysis was performed. It was found that the characteristic molecules of preadipocytes, Pdgfrb, Zfp423 and the related molecules of adipogenic differentiation, Pparg, Cebba and Fabp4 were highly expressed in ICAM-1⁺ cells (FIG. 1C). The results indicate that preadipocytes mainly exist in ICAM-1 positive stromal cells, and ICAM-1⁺ adipose stromal cells (CD31⁻CD45⁻Sca-1⁺PDGFR-α⁺) were rich in adipose stem cells and preadipocytes.

To further test whether ICAM-1⁺ adipose stem cells have the potential for spontaneous adipogenic differentiation, ICAM-1⁺ adipose stromal cells (CD31⁻CD45⁻Sca-1⁺PDGFR-α⁺) and ICAM-1⁻ adipose stromal cells (CD31⁻CD45⁻Sca-1⁺PDGFR-α⁺) were sorted, and their spontaneous adipogenic differentiation abilities were analyzed. Considering that spontaneous adipogenic differentiation may be the result of different cell populations interacting with each other through paracrine and other effects, the wild-type ICAM-1⁻ stromal cells and ICAM-1⁺ stromal cells from EGFP mice were mixed and cultured, so that different cell groups can be tracked without affecting their interactions.

The results show that during the initial growth period of the cells, the cells from the two sources show fibroblast morphology. In the later period of culture (day 8), spontaneous adipogenic differentiation occurred, and lipid droplets were accumulated in some cells. Interestingly, the vast majority of spontaneous adipogenic differentiation cells were EGFP⁺, indicating that ICAM-1⁺ adipose stromal cells may be adipose stem cells (FIG. 1D).

At the same time, reverse mixed culture was also carried out. Wild-type ICAM-1⁺ cells and EGFP of ICAM-1⁻ cells were mixed and cultured, and it was found that the spontaneous adipogenic differentiation cells were all ICAM-1⁺ cells. In addition, ICAM-1⁻ adipose stromal cells and ICAM-1⁺ adipose stromal cells were cultured separately, and studies have shown that only ICAM-1⁺ cells can spontaneously differentiate into adipocytes. These results indicate that ICAM-1⁺ adipose stromal cells are adipose stem cells with the ability of adipogenic differentiation.

### EXAMPLE 2

In Vivo Adipogenic Differentiation of ICAM-1⁺ Adipose Stem Cells

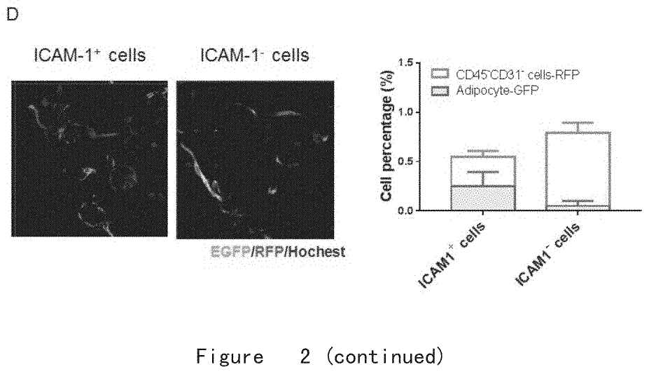

In order to fully verify whether ICAM-1⁺ adipose stromal cells are adipose stem cells, that is, whether they can generate mature adipocytes in vivo, Icaml-CreERT2 knock-in mice were made: using CRISPR/Cas9 technology, through homologous recombination, knocking the CreERT2 expression box into the ATG site of the ICAM-1 gene. In the Icaml-CreERT2 knock-in mice, the cells expressing ICAM-1 can express CreERT2. CreERT2 itself does not have recombinase activity and needs to be combined with Tamoxifen to activate its recombinase activity. mTmG tracer reports that in the absence of Cre recombinase, the cells and tissues from the whole body express the red fluorescent protein-tdTomato, located in the cell membrane. When Cre recombinase is present, the tdTomato expression sequence is deleted by recombination, and the downstream cell membrane-localized EGFP will be expressed, and the cells of its progeny will only express the cell membrane-localized EGFP. Therefore, we crossed Icaml-CreERT2 knock-in mice with tracer reporter mice mTmG, and treated newborn mice with Tamoxifen to activate the recombinase activity. The CreERT2 recombinase activated in ICAM-1⁺ cells cut the tdTomato expression sequence between the two loxP sites on the Rosa26 locus, and initiated the expression of EGFP on the following sequence, so that ICAM-1⁺ cells and their derived progeny cells all express EGFP (FIG. 2A).

The results show that when newborn mice were treated with Tamoxifen, EGFP adipocytes can be detected in the subcutaneous adipose tissue and visceral adipose tissue after adulthood (FIG. 2B). More importantly, when obese mice induced by high-fat diet are treated with Tamoxifen in the early stage, EGFP adipocytes can also be observed in the later period of obesity (FIG. 2C). Since adipocytes do not express ICAM-1, the above results indicate that ICAM-1⁺ adipose stem cells participate in the process of fat development and obesity through differentiation into mature adipocytes.

In addition, CD45⁻CD31⁻ICAM-1⁺ cells and CD45⁻CD31⁻ICAM-1⁻ cells were separated from the adipose tissue after hybridization of Icaml-CreERT2 knock-in mice and tracer reporter mice mTmG, and the above cells were mixed with matrigel (basement membrane matrix) and implanted into subcutaneous tissue in mice, treated with Tamoxifen, and adipose differentiation was observed. The research results show that ICAM-1⁺ cells can differentiate into EGFP-labeled adipocytes. This phenomenon is rare in matrigel implanted with ICAM-1⁻ cells (FIG. 2D), which shows that the ICAM-1 positive cells (such as CD45⁻CD31⁻ICAM-1⁺ adipose stem cells (adipose stromal cells)) of the present invention can be implanted in the body to differentiate into adipocytes spontaneously.

### EXAMPLE 3

Adipogenic Differentiation of ICAM-1⁺ Adipose Stem Cells Under Obesity

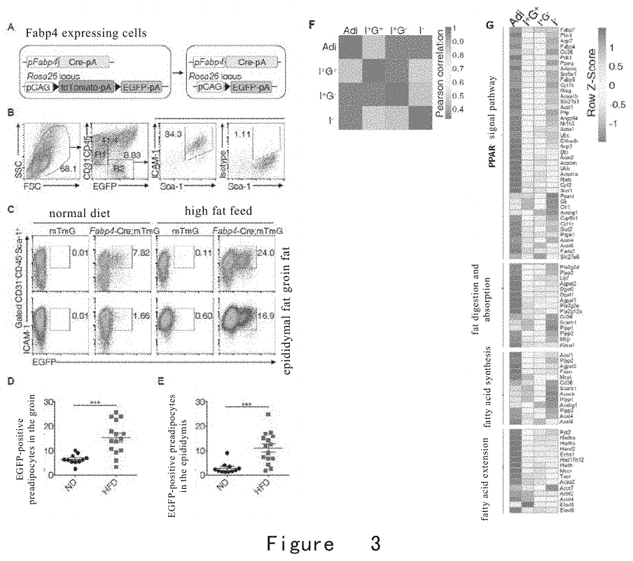

Next, we will discuss the correlation between this group of ICAM-1⁺ preadipocytes and obesity. For this purpose, another lineage tracing system was introduced. FABP4 is a characteristic molecule expressed when adipose stem cells are transformed into adipocytes. We hybridized Fabp4-Cre mice with mTmG tracer mice. In the obtained offspring mice, when the adipogenic differentiation of pre-adipocytes proceeds to the early adipocyte stage where Fabp4 is expressed, EGFP will be expressed (FIG. 3A). We fed Fabp4-Cre; mTmG mice with normal diet and high-fat diet, and analyzed the early differentiated adipocytes expressing EGFP⁺ in the stromal cells (CD45⁻CD31⁻Sca-1⁺) of the groin and epididymal adipose tissues.

The study has shown that under the normal feed, compared with Fabp4-Cre mice in the same littermate, Fabp4-Cre; mTmG adult mice had only a small amount of EGFP⁺ preadipocytes in adipose tissue, and mainly CD31⁻CD45⁻ Sca-1⁺ICAM-1⁺ (FIG. 3B) indicating that it is derived from ICAM-1⁺ adipose stem cells and still maintains the surface molecular phenotype of adipose stem cells. ICAM-1⁺ adipose stem cells participate in the normal replacement of adipocytes. Importantly, when these mice were induced to obesity with high-fat diet, there were a large number of early adipocytes expressing EGFP in both adipose tissues, and the surface molecular characteristics of ICAM-1⁺ adipose stem cells (CD31⁻CD45⁻Sca-1⁺ICAM-1⁺) were still maintained (FIG. 3C-D), which indicates that obesity induces the regeneration of adipocytes, and these newly differentiated adipocytes are mainly derived from ICAM-1⁺ adipose stem cells. At the same time, we have analyzed by immunofluorescence technology and have found these early EGFP⁺ICAM-1⁺ adipocytes in obese adipose tissues. They are all located around blood vessels and have the same localization as ICAM-1⁺ adipose stem cells (FIG. 3E).

In order to further identify these ICAM-1⁺EGFP⁺ cells, we sorted mature adipocytes, ICAM-1⁺EGFP⁺, ICAM-1⁺EGFP⁻ and ICAM-1⁻cell subsets from obese mice for RNA-seq analysis. We have found that the gene expression profile of the ICAM-1⁺EGFP⁺ subset is very similar to that of ICAM-1⁺EGFP⁻ subset, with a correlation coefficient of 0.98 (FIG. 3F). Compared with other subsets, ICAM-1⁺EGFP⁺ cells have similar gene expression patterns to adipocytes (FIG. 3F), especially when focusing on genes in adipocyte characteristic signaling pathways (FIG. 3G). In the expression of these adipocyte characteristic genes, adipocytes have the highest correlation with ICAM-1⁺EGFP⁺, followed by ICAM-1⁺EGFP⁻ cells, having the lowest correlation with ICAM-1⁻. These results indicate that ICAM-1⁺EGFP⁺ cells are the intermediate product of adipogenic differentiation, and are derived from ICAM-1⁺EGFP⁻ adipose stem cells.

### EXAMPLE 4

ICAM-1 Negatively Regulates the Terminal Differentiation of Preadipocytes

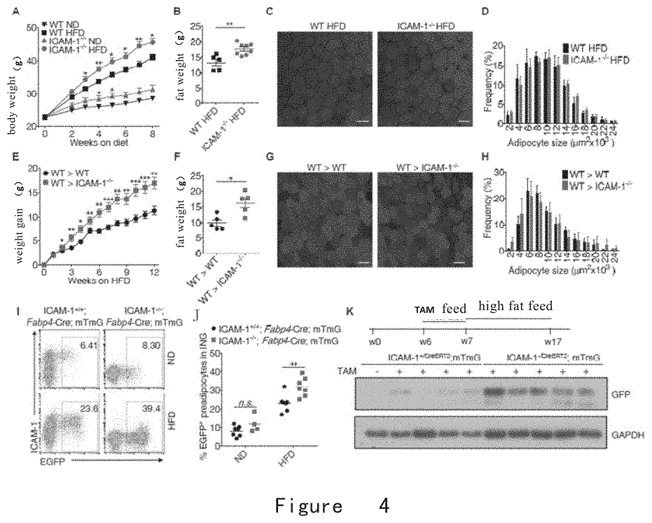

Based on the above research, it has been proved that ICAM-1 is expressed on adipose stem cells and preadipocytes and adipogenic differentiation was performed when obesity occurs. However mature adipocytes do not express ICAM-1, and the expression of ICAM-1 is gradually decreased during adipogenic differentiation. This expression characteristic is very similar to the that of characteristic genes Pref-1 and GATA2/GATA3 of preadipocytes. These genes have the function of resisting adipogenic differentiation and maintaining the undifferentiated state of preadipocytes. Based on this, it is speculated that ICAM-1 can play the same regulatory role. Studies have shown that compared with wild-type mice, ICAM-1^{-/-} mice have significantly increased body weight and adipose tissue weight regardless of normal diet or high-fat diet, and the increase in adipose tissues does not depend on the increase in the volume of adipocyte (FIG. 4A-D). Since ICAM-1 is expressed on immune cells, in order to rule out the effect of ICAM-1 deficiency in immune cells on obesity, we conducted bone marrow replacement experiments and have found that even if the immune cells of ICAM-1^{-/-} mice are replaced with wild-type mice immune cells, they are still more prone to obesity (FIG. 4E-F). Since fat proliferation includes two modes, cell enlargement and increase, we have found through analysis that the size of adipocyte in ICAM-1^{-/-} mice does not increase significantly (FIG. 4G-H), indicating that the increase in the number of adipocytes plays an important role in the obesity, which is a result of the excessive differentiation of adipose stem cells.

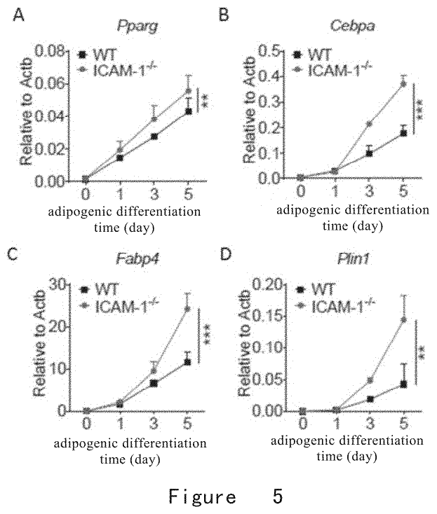

In order to determine the contribution of increased number of adipocytes to obesity, we hybridized ICAM-1^{-/-} mice with Fabp4-Cre; mTmG mice and have found that compared with ICAM-1^{-/-}; Fabp4-Cre; mTmG littermate mice, ICAM-1^{-/-}; Fabp4-Cre; mTmG mice have significantly increased EGFP⁺ adipogenic differentiation intermediate cells (FIG. 4I-K), indicating that the lack of ICAM-1 can promote the adipogenic differentiation process of adipose stem cells in vivo. Compared with wild-type adipose stem cells, ICAM-1^{-/-} primary pre-adipocytes are differentiated faster (FIG. 4H), and adipogenic differentiation genes (including Pparg, Cebpa, Fabp4 and Plinl) are significantly increased (FIG. 5A-D). Therefore, ICAM-1 negatively regulates the terminal differentiation of adipose stem cells.

### EXAMPLE 5

ICAM-1 Maintains the Undifferentiated State of Adipose Stem Cells Through Rho and ROCK

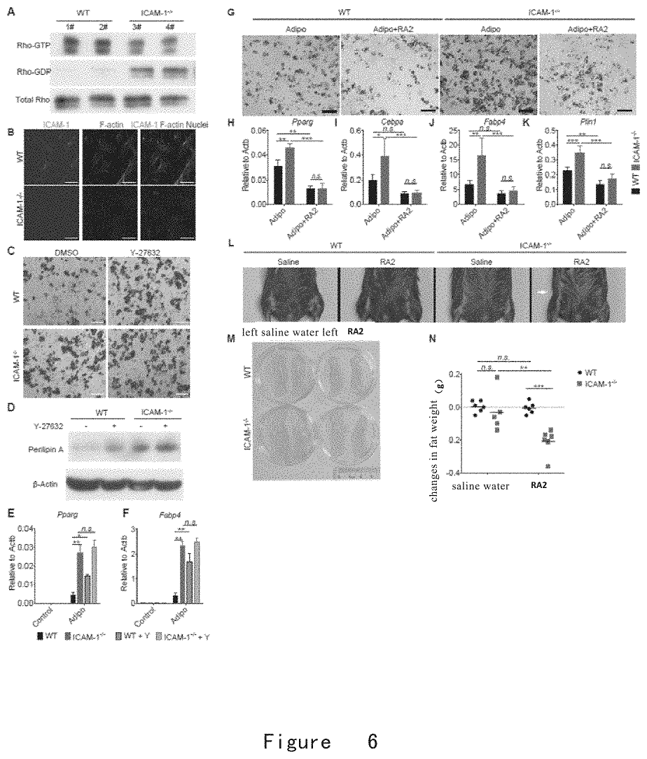

Next, we explored the molecular mechanism of ICAM-1 controlling adipogenic differentiation. A very important component in the downstream signal of ICAM-1 is the small GTPase Rho. We have found that the activated form of Rho (Rho-GTP) in ICAM-1^{-/-} preadipocytes is significantly less than that in wild-type precursor cells, and the inactive Rho-GDP is higher than that in wild-type precursor cells (FIG. 6A). Activated Rho can regulate the formation of tension fibers in cells through ROCK. Through fluorescence immunoassay of F-actin, we have found that there are a large number of tightly structured tension fibers in wild-type precursor cells, and there are co-localization of the fibre packing of F-actin and ICAM-1 clusters; while in the precursor cells of ICAM-1^{-/-}, the density of tension fibers is significantly lower than that of wild-type stromal cells, and the structure is loose, with few fiber bunching (FIG. 6B). This shows that ICAM-1 can activate Rho and ROCK in preadipocytes, and plays an important role in the assembly of tension fibers and the construction of cytoskeleton.

Rho and ROCK can negatively regulate adipogenic differentiation in a cytoskeleton-dependent or insulin signal-dependent manner, and our RNA-seq data also supports the role of Rho GTPase in adipose differentiation. In order to test whether Rho and ROCK are involved in the inhibitory effect of ICAM-1 on adipogenic differentiation of adipose stem cells, we used ROCK inhibitor Y-27632 to treat adipose stem cells respectively. We have found that compared with the DMSO-treated group, Y-27623 can significantly accelerate the adipogenic differentiation of wild-type adipose stem cells, but has no obvious effect on the adipogenic differentiation of ICAM-1^{-/-} adipose stem cells (FIG. 6C). At the same time, we have analyzed the expression level of Perilipin A, a characteristic protein of mature adipocytes, and found that Y-27632 can significantly increase the expression level of this protein in wild-type adipose stem cells, but has little effect in ICAM-1^{-/-} adipose stem cells (FIG. 6C). In addition, inhibiting ROCK can significantly increase the expression of adipogenic differentiation-related proteins and genes in wild-type mice of adipose stem cells, including Perilipin A, Pparg and Fabp4, while the effect is not obvious in ICAM-1^{-/-} mice-derived adipose stem cells (FIG. 6D-F). Therefore, ICAM-1 inhibits the adipogenic differentiation of adipose stem cells through the Rho-ROCK pathway.

In order to verify whether Rho GTPase activity can reverse the excessive adipogenic differentiation caused by ICAM-1 deletion, we used the Rho agonist Rho activator II (RA2), which can constitutively activate RhoGTPase. We have found that RA2 has significantly inhibited the adipogenic differentiation ability of ICAM-1^{-/-} adipose stem cells, but the effect on wild-type adipose stem cells is not obvious (FIG. 6G). Consistent with this, activation of Rho GTPase in ICAM-1^{-/-} precursor cells has resulted in an extensive reduction of adipogenic differentiation genes, including Pparg, Cebpa, Fabp4, and Plinl, while in wild-type cells, only Pparg and Fabp4 are significantly changed by Rho GTPase activation (FIG. 6H-K). It is important that the difference in the expression of adipogenic differentiation genes between wild-type and ICAM-1^{-/-} cells is eliminated by Rho

GTPase activation (FIG. 6H-K). These results confirm that ICAM-1 regulates adipogenic differentiation through Rho GTPase.

In order to test whether ICAM-1 regulates adipogenic differentiation through Rho GTPase in vivo, we injected RA2 locally into the right inguinal fat pad of mice, and compared it with the left fat pad to demonstrate the effect of local activation of Rho GTPase. After 10 weeks of RA treatment on mice fed with high-fat diet, we have found that the excessive adipogenic differentiation of ICAM-1^{-/-} mice is weakened, and the inguinal fat pads on both sides are asymmetric (FIG. 6L). This asymmetry is not observed in RA2-treated WT mice and PBS-treated mice (FIG. 6L). We have collected these adipose tissues for weighing, and found that RA2 can significantly reduce fat weight in ICAM-1^{-/-} but not WT mice (FIG. 6M-N).

### EXAMPLE 6

ICAM-1 Negatively Regulates the Differentiation of Human Preadipocytes

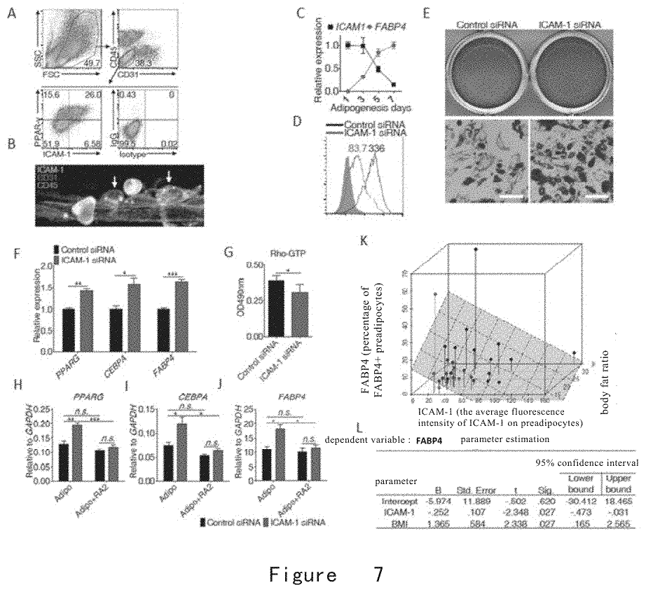

First, the expression content of ICAM-1 in human adipose tissue was analyzed. Currently, there is no recognized characteristic molecule of human preadipocytes. We have found that ICAM-1 is widely expressed in human CD31⁻CD45⁻ adipose stromal cells (FIG. 7A). These ICAM-1⁺ cells, like adipose tissue in mice, are mainly located around blood vessels (FIG. 7B). To test the regulatory effect of ICAM-1 on human adipose stem cells, we isolated human primary adipose stromal cells for adipogenic differentiation induction. We have found that consistent with mice, the expression level of ICAM1 in human preadipocytes is gradually decreased with adipogenic differentiation (FIG. 7C). When the expression of ICAM1 is knocked down with siRNA (FIG. 7D), the adipogenic differentiation of human preadipocytes is significantly enhanced (FIG. 7E), and the expression of adipogenic genes including PPARG, CEBPA and FABP4 is significantly increased (FIG. 7F), indicating that ICAM-1 has a negative regulatory effect on the adipogenic differentiation of human adipose stem cells. It is worth noting that knockdown of ICAM-1 resulted in a decrease in the Rho GTPase activity of human adipose stem cells (FIG. 7G). When RA2 is used to treat human adipose stem cells during differentiation, the enhancement of adipogenic differentiation of ICAM-1 knock-down cells is eliminated (FIG. 7H-J). Therefore, ICAM-1 also has the ability to negatively regulate the terminal differentiation of human adipose stem cells.

In order to test the physiological role of ICAM-1 on human preadipocytes, we collected human adipose tissue samples from patients undergoing plastic surgery, and analyzed the expression levels of ICAM-1 and FABP4 on CD31⁻CD45⁻ adipose stromal cells by flow cytometry. The expression level of ICAM-1 is significantly correlated with the subject's body fat ratio (BMI) (FIG. 7K), which is similar to the observations in mice. We used linear regression analysis to test the correlation between the expression level of ICAM-1 and the proportion of FABP4⁺ preadipocytes. In view of the strong correlation between BMI and the expression of ICAM-1, we introduced a linear model in which the interaction term of BMI and ICAM-1 MFI was modified. On this basis, we have found that the proportion of FABP4⁺ preadipocytes is significantly negatively correlated with the expression level of ICAM-1 (FIG. 7K-L), indicating that ICAM-1 has a negative regulatory effect on the in vivo adipogenic differentiation of human preadipocytes.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A method for promoting the differentiation of an adipose stem cell into an adipocyte or remodeling of adipose tissues, comprising administering an ICAM-1 inhibitor to a subject in need thereof;
preferably, the adipose stem cell is an ICAM-1 positive adipose stromal cell.

2. The method of claim 1, wherein the adipose stem cell expresses a regulatory gene for adipogenic differentiation, wherein the regulatory gene for adipogenic differentiation is selected from the group consisting of: Pparg, Cebpa, Cebpb, Cebpg, Gata2, Gata3, Irs1, Pparg, Cebba and Fabp4, and a combination thereof.

3. (canceled)

4. A method for inhibiting the differentiation of an adipose stem cell into an adipocyte, comprising administering an ICAM-1 or a promoter thereof to a subject in need thereof or maintaining the ICAM-1 expression level of the adipose stem cell.

5. A method for preparing an adipocyte, comprising the steps of:
(a) providing an ICAM-1 positive adipose stromal cell;
(b) culturing the adipose stromal cell under conditions suitable for adipocyte differentiation, thereby obtaining a cell population containing a differentiated adipocyte; and
(c) isolating the adipocyte from the cell population.

6. The method of claim 5, wherein the adipose stromal cell is a CD45-CD31-Sca-1+PDGFR-α+ICAM-1+ cell or a CD45-CD31-ICAM-1+ cell.

7. The method of claim 5, wherein in step (b) and step (c), detecting the expression level of ICAM-1 to determine the degree of differentiation of the adipose stromal cell into the adipocyte in the cell population.

8. (canceled)

9. Use of ICAM-1 or a detection reagent thereof for preparing a detection kit for (a) the detection of an adipose stem cell, and/or (b) the determination of the risk of obesity in a test subject.

10. A diagnostic kit, containing a container, wherein the container contains ICAM-1 or a detection reagent thereof; and a label or instructions, wherein the label or instructions indicate that the kit is used for (a) detecting an adipose stem cell, and/or (b) determining the risk of obesity in the test subject.

11. A method for the remodeling of an adipose tissue, comprising administering a stromal cell, which is an ICAM-1 positive stromal cell isolated from an adipose tissue to a subject in need thereof.

12. A method for determining the risk of obesity in a test subject, comprising the steps of:
(a) providing a sample from a test subject;
(b) determining the proportion of ICAM-1⁺ cells in the sample as A1;
(c) comparing the A1 in step (b) with the proportion A0, of ICAM-1⁺ cells in the normal population sample, and if A1 is significantly higher than A0, it is indicated that the test subject has a high risk of obesity.
